(19) United States
(12) Patent Application Publication
(10) Pub. No.: US 2019/0167982 A1
(43) Pub. Date: Jun. 6, 2019

(54) STIMULATION HEALTH CARE MODULE

(71) Applicant: Y-BRAIN INC., Daejeon (KR)

(72) Inventor: Kiwon LEE, Gyeonggi-do (KR)

(21) Appl. No.: 15/833,016

(22) Filed: Dec. 6, 2017

(51) Int. Cl. A61N 1/20; A61N 1/02

(52) U.S. Cl. CPC A61N 1/20; A61N 1/025

(57) ABSTRACT

A stimulation health care module includes a control unit for storing inputted stimulation strength, stimulation duration and stimulation frequency in internal memory; and a current providing unit for providing the control unit with current corresponding to the stimulation strength, the stimulation duration and the stimulation frequency, in which the control unit executes stimulation corresponding to the stimulation strength, the stimulation duration and the stimulation frequency using the current provided by the current providing unit.

### BACKGROUND

The present invention relates to a stimulation health care module, and more specifically, to a stimulation health care module, which can store stimulation strength, stimulation duration and stimulation frequency inputted through a management station and easily execute stimulation at a stimulation location (e.g., home of a patient) convenient to execute stimulation corresponding to the stimulation strength, the stimulation duration and the stimulation frequency.

Presently, brains are stimulated in a variety of methods including deep electrical stimulation, Transcranial Magnetic Stimulation (TMS), Transcranial Electrical Stimulation (TES), Transcranial Direct Current Stimulation (tDCS) and Transcranial Random Noise Stimulation (tRNS).

Particularly, the Transcranial Direct Current Stimulation is utilized as a method of stimulating a brain in a rehabilitation or neuropsychiatry clinic, and it is reported to be effective for improving cognitive ability and treating mental disorders such as depression, Attention Deficit Hyperactivity Disorder (ADHD) and the like.

A background technique of the present invention is disclosed in US Laid-opened Patent No. US 2009/0287108.

### SUMMARY OF THE INVENTION

Therefore, the present invention has been made in view of the above problems, and it is an object of the present invention to provide a stimulation health care module, which can store stimulation strength, stimulation duration and stimulation frequency inputted through a management station and easily execute stimulation at a remote location (e.g., home of a patient) convenient to execute stimulation corresponding to the stimulation strength, the stimulation duration and the stimulation frequency.

To accomplish the above object, according to one aspect of the present invention, there is provided a stimulation health care module including: a control unit for storing inputted stimulation strength, stimulation duration and stimulation frequency in internal memory; and a current providing unit for providing the control unit with current corresponding to the stimulation strength, the stimulation duration and the stimulation frequency, in which the control unit may execute stimulation corresponding to the stimulation strength, the stimulation duration and the stimulation frequency using the current provided by the current providing unit.

In the stimulation health care module according to an embodiment of the present invention, the stimulation strength may be strength of DC current, the stimulation duration may be a time period of sustaining the strength of DC current, and the stimulation frequency may be the number of times of repeating the time period of sustaining the strength of DC current.

The stimulation health care module according to an embodiment of the present invention may further include a real-time clock for transferring real-time to the control unit, and if the number of times of stimulation is two or more, the control unit may control to execute second and subsequent stimulations after a minimum time period is elapsed using the real-time.

The stimulation health care module according to an embodiment of the present invention may further include an alarm unit controlled by the control unit to inform a recommended time desirable to execute the stimulation using the real-time.

The stimulation health care module according to an embodiment of the present invention may further include a memory unit controlled by the control unit to store a time when the stimulation has been executed using the real-time.

The stimulation health care module according to an embodiment of the present invention may further include a display unit controlled by the control unit to separately display the number of times of stimulation that has been executed already and the number of times of stimulation that has not been executed yet or to display remaining stimulation duration.

### BRIEF DESCRIPTION OF THE DRAWINGS

FIG. 1 is a block diagram showing the configuration of a stimulation health care module according to an embodiment of the present invention.

FIG. 2 is a view showing a display unit of a stimulation health care module according to an embodiment of the present invention.

### DETAILED DESCRIPTION OF THE PREFERRED EMBODIMENT

Specific matters of other embodiments are included in the detailed description and the drawings.

Advantages and features of the present invention and a method of achieving the same will be more clearly understood from the embodiments described below with reference to the accompanying drawings. However, the present invention is not limited to the following embodiments but may be implemented in various different forms. The embodiments are provided merely to complete disclosure of the present invention and to fully provide a person having ordinary skill in the art to which the present invention pertains with the category of the invention. The invention is defined only by the category of the claims. Wherever possible, the same reference numbers will be used throughout the specification to refer to the same or like parts.

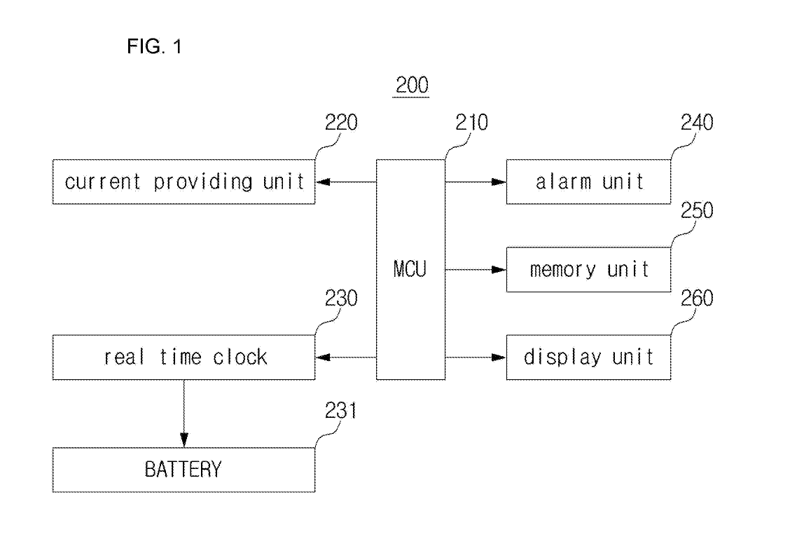

A control unit 210 of a stimulation health care module 200 according to an embodiment of the present invention is configured of a Micro Controller Unit (MCU) as shown in FIG. 1 and stores inputted stimulation strength, stimulation duration and stimulation frequency in internal memory.

A current providing unit 220 includes a current sensor and an operational amplifier and provides the control unit 210 with current corresponding to the stimulation strength, the stimulation duration and the stimulation frequency, and the control unit 210 executes stimulation corresponding to the stimulation strength, the stimulation duration and the stimulation frequency using the current provided by the current providing unit 220.

Here, the stimulation strength is strength of DC current, for example, DC current of about 0 to 2 mA, the stimulation duration is a time period of sustaining the strength of DC current, for example, a time period of about ten to thirty minutes, and the stimulation frequency is the number of times of repeating the time period of sustaining the strength of DC current, for example, once a day for thirty days.

Meanwhile, the stimulation health care module further includes a real-time clock 230 for transferring real-time to the control unit 210, and if the number of times of stimulation is two or more, the control unit 210 controls to execute second and subsequent stimulations after a minimum time period is elapsed using the real-time. Here, the minimum time period may be set to be, for example, four hours or more. For example, if the stimulation strength is DC current of 1 mA, the stimulation duration is twenty minutes, the stimulation frequency is once a day for thirty days, the minimum time period is four hours, and the first stimulation is executed at 23:00 on Sep. 1, 2016, the second stimulation can be executed only after 03:00 on Sep. 2, 2016.

In addition, the present invention may be configured to record and manage corresponding information if the stimulation is not executed during certain one day. In other words, if the stimulation is not executed between 00:00 and 24:00 on September 6^{th} although it is set to execute stimulation thirty times for thirty days between September 1^{st} and 30^{th}, the control unit 210 may record and manage corresponding information. In this case, although the overall stimulation schedule is preferably set to finish on September 30^{th} regardless of the number of times of stimulation that has not been executed, the present invention is not necessarily limited thereto.

Meanwhile, the stimulation health care module according to an embodiment of the present invention may be provided with a separate battery 231 and supply power to the real-time clock 230.

In addition, the stimulation health care module further includes an alarm unit 240 controlled by the control unit 210 to inform a recommended time desirable to execute the stimulation using the real-time.

For example, when the stimulation strength is DC current of 1 mA, the stimulation duration is twenty minutes, and the stimulation frequency is once a day for thirty days, a recommended time is informed at 21:00 every day through the alarm unit 240 so that stimulation may be executed at the same time for thirty days.

In addition, the stimulation health care module further includes a memory unit 250 controlled by the control unit 210 to store a time when the stimulation has been executed using the real-time. Meanwhile, remaining stimulation duration is stored in the memory unit 250 when the stimulation is stopped due to unexpected reasons, and if the stimulation is executed at a later time, the control unit 210 executes remaining stimulation using the remaining stimulation duration stored in the memory unit 250.

For example, when the stimulation strength is DC current of 1 mA, the stimulation duration is twenty minutes, and the stimulation frequency is once a day for thirty days, a time when the stimulation has been executed is stored in the memory unit 250 using the real-time.

Meanwhile, the stimulation health care module further includes a display unit 260 controlled by the control unit 210 to separately display the number of times of stimulation that has been executed already and the number of times of stimulation that has not been executed yet or to display remaining stimulation duration.

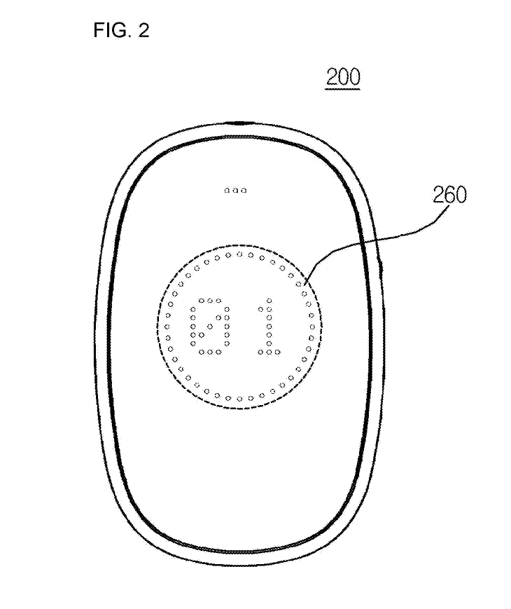

Referring to FIG. 2, for example, if the stimulation strength is DC current of 1 mA, the stimulation duration is twenty minutes, the stimulation frequency is once a day for thirty days, and the first stimulation is executed on Sep. 1, 2016, ‘1’, which is the number of times of stimulation that has already been executed, is displayed by turning on one blue Light Emitting Diode (LED), and ‘29’, which is the number of times of stimulation that has not been executed yet, is displayed on the display unit 260 by turning on twenty-nine red LEDs, or if stimulation is executed while ‘20’ is displayed on the display unit 260 as the remaining stimulation duration, it is displayed as 19, 18, 17, . . . , 2, 1, 0.

Alternatively, it may be configured such that although information on the remaining number of times of stimulation is displayed in normal times using numeric information of the display unit 260, information on the remaining stimulation duration may be displayed only when the stimulation is executed.

For reference, when the stimulation health care module of the present invention is used for treatment purpose in a clinic, not for home treatment, display of the information on the remaining number of times of stimulation may not be necessary, and accordingly, in this case, it may be configured not to display any information or to display unmeaningful information such as 0 or the like in normal times.

The stimulation health care module according to an embodiment of the present invention can store stimulation strength, stimulation duration and stimulation frequency inputted through a management station and easily execute stimulation at a remote location (e.g., home of a patient) convenient to execute stimulation corresponding to the stimulation strength, the stimulation duration and the stimulation frequency.

A person having ordinary skill in the art to which the present invention pertains may understand that the present invention may be embodied in other specific forms without changing its technical spirit or essential features of the present invention.

Therefore, the embodiments described above are to be considered in all respects as illustrative and not restrictive.

The scope of the present invention is represented by the claims rather than the detailed description described above, and it is to be interpreted that the meaning and scope of the claims and all the changes or modified forms derived from the equivalents thereof come within the scope of the present invention.

What is claimed is:

1. A stimulation health care module comprising:
a control unit for storing inputted stimulation strength, stimulation duration and stimulation frequency in internal memory; and
a current providing unit for providing the control unit with current corresponding to the stimulation strength, the stimulation duration and the stimulation frequency, wherein
the control unit executes stimulation corresponding to the stimulation strength, the stimulation duration and the stimulation frequency using the current provided by the current providing unit.

2. The module according to claim 1, wherein the stimulation strength is strength of DC current, the stimulation duration is a time period of sustaining the strength of DC current, and the stimulation frequency is the number of times of repeating the time period of sustaining the strength of DC current.

3. The module according to claim 2, further comprising a real-time clock for transferring real-time to the control unit, wherein if the number of times of stimulation is two or more, the control unit controls to execute second and subsequent stimulations after a minimum time period is elapsed using the real-time.

4. The module according to claim 3, further comprising an alarm unit controlled by the control unit to inform a recommended time desirable to execute the stimulation using the real-time.

5. The module according to claim 3, further comprising a memory unit controlled by the control unit to store a time when the stimulation has been executed using the real-time.

6. The module according to claim 3, further comprising a display unit controlled by the control unit to separately display the number of times of stimulation that has been executed already and the number of times of stimulation that has not been executed yet or to display remaining stimulation duration.
